(19) United States
(12) Patent Application Publication
(10) Pub. No.: US 2026/0364779 A1
(43) Pub. Date: Nov. 19, 2026

(54) MAGNETIC CONTROL SYSTEM FOR MICROROBOT MANIPULATION

(71) Applicant: Applied Research Associates, Inc., Albuquerque, NM (US)

(72) Inventors: Louis William Rogowski, Randolph, VT (US); Tobias William Cooke, Albuquerque, NM (US); Justin Andrew Wood, Alexandria, VA (US)

(21) Appl. No.: 19/676,412

(22) Filed: May 13, 2026

Related U.S. Application Data
(60) Provisional application No. 63/869,782, filed on Aug. 25, 2025.
(60) Provisional application No. 63/806,899, filed on May 16, 2025.

(51) Int. Cl. B25J 9/00; B25J 9/16; H01F 7/18; H01F 38/14

(52) U.S. Cl. CPC B25J 9/0009; B25J 9/1694; H01F 7/18; H01F 38/14; H01F 2038/143

(57) ABSTRACT

The present disclosure provides a magnetic control system for microrobots. The system includes a power supply unit configured to provide electrical power, a signal generator configured to generate control signals, and an amplifier electrically coupled to the power supply unit and the signal generator. The amplifier is configured to amplify the control signals. A coil assembly is electrically coupled to the amplifier and configured to generate magnetic fields based on the amplified control signals. A cooling system is thermally coupled to the coil assembly and configured to dissipate heat generated by the coil assembly. The system enables precise control of magnetic fields for manipulating microrobots while managing heat generation during operation.

### CROSS-REFERENCE TO RELATED APPLICATIONS

This patent application is a non-provisional application claiming priority benefit, with regard to all common subject matter, of U.S. Provisional Patent Application No. 63/869,782, filed Aug. 25, 2025, entitled “MAGNETIC CONTROL SYSTEM FOR MICROROBOT MANIPULATION,” and U.S. Provisional Patent Application No. 63/806,899, filed May 16, 2025, entitled “MAGNETIC CONTROL SYSTEM FOR MICROROBOT MANIPULATION.” The above-referenced applications are hereby incorporated by reference in their entirety into the present application

### FIELD OF INVENTION

The present disclosure relates to magnetic control systems for microrobots, and more particularly to a compact, portable magnetic control assembly with integrated cooling and high-fidelity signal generation for precise manipulation of microrobots.

### BACKGROUND

Microrobots are artificial composite structures capable of performing tasks at the microscopic scale. These tiny structures, often measuring less than a millimeter in size, have garnered interest across various fields due to their potential for precise manipulation and interaction in low Reynolds number environments. Microrobots typically incorporate magnetic materials into their structure, allowing them to be controlled and moved using external magnetic fields.

Despite their potential, controlling these microrobots with precision remains challenging. The small scale at which they operate introduces complexities in terms of power delivery, communication, and navigation. Environmental factors such as fluid dynamics and surface interactions can significantly impact the behavior of microrobots with magnetic properties. Additionally, generating and manipulating magnetic fields to effectively control microrobots requires specialized equipment and techniques.

Conventional magnetic control systems for microrobots present significant spatial challenges, often requiring extensive laboratory infrastructure. These systems typically consist of bulky electromagnetic coils, substantial power supplies, cooling equipment, and control electronics that occupy considerable physical space. The large footprint of these setups restricts microrobot research and applications to well-equipped laboratory environments with dedicated facilities. This spatial requirement creates barriers to field deployment, limits accessibility for smaller research institutions with space constraints, and hinders the adoption of microrobot technology in practical applications outside controlled laboratory settings. Furthermore, the substantial infrastructure requirements translate to higher implementation costs and reduced scalability for multi-site deployments.

As research in micro-robotics progresses, there is a growing need for versatile and reliable control systems that can support a wide range of applications while addressing the spatial limitations of current technologies. Such systems would ideally combine precise magnetic field generation, advanced cooling capabilities, and user-friendly interfaces within a compact form factor that enables deployment beyond traditional laboratory environments. The development of portable, integrated magnetic control systems represents a critical advancement that could significantly expand the practical applications of microrobot technology across diverse fields.

### SUMMARY

This summary is provided to introduce a selection of concepts in a simplified form that are further described below in the detailed description. This summary is not intended to identify key features or essential features of the claimed subject matter, nor is it intended to be used to determine the scope of the claimed subject matter.

According to an aspect of the present disclosure, a compact and portable magnetic control system for microrobots is provided. The magnetic control system includes a power supply unit configured to provide electrical power. The system also includes a signal generator configured to generate control signals. The system further includes an amplifier electrically coupled to the power supply unit and the signal generator; the amplifier is configured to amplify the control signals. The system also includes a coil assembly electrically coupled to the amplifier; the coil assembly is configured to generate magnetic fields based on the amplified control signals. The system further includes a cooling system thermally coupled to the coil assembly; the cooling system is configured to dissipate heat generated by the coil assembly.

According to other aspects of the present disclosure, the magnetic control system may include one or more of the following features. The power supply unit may include multiple power supplies configured to provide bipolar power. The signal generator may include a microcontroller programmed to generate arbitrary waveforms. The amplifier may include linear amplifiers configured in a bridge-tied load configuration. The coil assembly may include multiple coil pairs arranged orthogonally. The cooling system may include water blocks thermally coupled to the coils, a pump, and a heat exchanger.

The system may further include a control interface configured to receive user inputs and control the signal generator. The control interface may include software running on an external computer, the software configured to communicate with the signal generator via a USB interface. The system may be configured to generate both static and rotating magnetic fields. The system may be configured to generate magnetic field gradients. The system may be configured to automatically adjust capacitance to optimize power factor based on a function of operating frequency and electrical properties of the connected coil pairs.

According to another aspect of the present disclosure, a method of controlling microrobots using a magnetic control system is provided. The method includes generating control signals using a signal generator. The method also includes amplifying the control signals using an amplifier. The method further includes generating magnetic fields using a coil assembly based on the amplified control signals. The method also includes dissipating heat generated by the coil assembly using a cooling system.

According to other aspects of the present disclosure, the method may include one or more of the following features. Generating control signals may include generating arbitrary waveforms using a microcontroller. Amplifying the control signals may include using linear amplifiers in a bridge-tied load configuration. Generating magnetic fields may include generating both static and rotating magnetic fields. The method may further include receiving user inputs via a control interface and adjusting the control signals based on the user inputs. The method may also include automatically adjusting capacitance to optimize power factor based on a function of operating frequency and the electrical properties of the connected coil pairs.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates a block diagram of a magnetic control system, according to aspects of the present disclosure.

FIG. 2 illustrates a detailed view of signal generation and amplification components, according to an embodiment.

FIG. 3 illustrates an exploded view of a coil assembly for generating magnetic fields, according to aspects of the present disclosure.

FIG. 4A illustrates an isometric view of a magnetic coil assembly, according to an embodiment.

FIG. 4B illustrates a section view of a magnetic coil assembly, according to aspects of the present disclosure.

FIG. 4C and FIG. 4D illustrate isometric views of a water-cooled coil assembly, according to an embodiment.

FIG. 4E illustrates an exploded view of a water-cooled coil assembly, according to aspects of the present disclosure.

FIG. 4F illustrates an exploded view of a water-cooled coil assembly, according to aspects of the present disclosure.

FIG. 4G illustrates an exploded view of a water-cooled coil assembly, according to aspects of the present disclosure.

FIG. 4H illustrates a cutaway view of a water-cooled coil assembly, according to aspects of the present disclosure.

### DETAILED DESCRIPTION

The following detailed description references the accompanying drawings that illustrate specific embodiments in which the present disclosure can be practiced. The embodiments are intended to describe aspects of the present disclosure in sufficient detail to enable those skilled in the art to practice the present disclosure. Other embodiments can be utilized, and changes can be made without departing from the scope of the present disclosure. The following detailed description is, therefore, not to be taken in a limiting sense. The scope of the present disclosure is defined only by the appended claims, along with the full scope of equivalents to which such claims are entitled. Unless otherwise expressly stated, numerical values, dimensions, materials, configurations, and operating parameters described herein are provided as illustrative examples and are not intended to be limiting. One skilled in the art will recognize that variations, substitutions, and modifications may be made without departing from the scope of the present disclosure.

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments but is not necessarily included. Thus, the technology can include a variety of combinations and/or integrations of the embodiments described herein.

As described herein, a compact and portable microrobot control system utilizes magnetic fields to manipulate the movement and behavior of microscale robots. The microrobots incorporate magnetic materials within their structure, enabling external control through applied magnetic fields. The magnetic materials may consist of ferromagnetic structures such as iron oxide (Fe₃O₄, Fe₂O₃) nanoparticles with diameters ranging from 10-100 nm, neodymium-iron-boron (NdFeB) microparticles, cobalt-based alloys, or nickel-iron permalloy compositions. These particles may be uniformly distributed throughout the microrobot's composition matrix. The magnetic particles serve multiple functions, including enabling directional locomotion through field-induced torque, facilitating position control via magnetic field gradients, and allowing rotational control through oscillating or rotating fields. The incorporation of magnetic particles offers advantages such as wireless actuation without onboard power sources, precise spatial control with six degrees of freedom, and the ability to operate in various environments, including biological fluids, industrial processes, or environmental remediation scenarios.

By modulating the strength, direction, and gradients of the applied magnetic fields, the system may induce forces and torques on the microrobots, enabling controlled locomotion, rotation, and actuation. The magnetic control system may generate both static and dynamic magnetic fields to achieve various control objectives. Static fields may be used to hold microrobots in place, spatially orient the microrobots’ magnetic dipoles, or help guide them along predetermined paths. Dynamic fields, including rotating fields, may enable more complex maneuvers such as propulsion through fluid environments or controlled tumbling motions. By generating magnetic field gradients, the system may exert pulling or pushing forces on the microrobots, allowing for precise positioning and navigation within the working space.

In some embodiments, the magnetic control system may further include one or more movable magnetic field sources configured to modulate the magnetic field applied to the microrobots or to a sample within the working space. The movable magnetic field sources may include electromagnets, permanent magnets, or combinations thereof mounted to translation stages, rotary stages, robotic actuators, linear actuators, gimbals, rails, articulated arms, or other positioning mechanisms. The positioning mechanisms may selectively move, translate, rotate, tilt, or reposition the magnetic field sources relative to the working space to dynamically vary magnetic field strength, orientation, spatial distribution, or magnetic field gradients experienced by the microrobots or sample.

For example, one or more permanent magnets may be translated toward or away from the working space to increase or decrease magnetic field intensity. In other embodiments, one or more magnets may be rotated about one or more axes to alter magnetic field direction or generate rotating magnetic field patterns. In further embodiments, movable permanent magnets may operate in conjunction with electromagnet coils such that coarse magnetic field generation is provided by the permanent magnets while fine magnetic field modulation is provided by the electromagnets.

Components of the compact magnetic control system include a housing, a power supply unit, a signal generator, an amplifier, a coil assembly, and a cooling system. The power supply unit may include multiple power supplies configured to provide bipolar power, enabling the generation of alternating current signals, and therefore alternating magnetic fields or static fields of any polarity along any axis. The signal generator, which may incorporate a microcontroller, allows for the creation of arbitrary waveforms to produce complex magnetic field patterns. The amplifier, utilizing linear amplifiers in a bridge-tied load configuration, amplifies the control signals to drive the coil assembly.

The coil assembly forms the core of the magnetic field generation system. It may include multiple coil pairs arranged orthogonally to create a three-dimensional control volume. This arrangement allows for the generation of magnetic fields along different axes, enabling comprehensive spatial control of the microrobots. The cooling system, featuring water blocks thermally coupled to the coils, a pump, and a heat exchanger, helps maintain thermal stability during operation, ensuring consistent and precise magnetic field generation as well as extending the possible operating time.

In some embodiments, the magnetic control assembly may include one or more electromagnets, permanent magnets, or combinations thereof configured to generate magnetic fields within the working space. The electromagnets may include air-core coils, ferromagnetic-core coils, Helmholtz coils, Maxwell coils, gradient coils, or other magnetic field generation structures. Permanent magnets may include rare-earth magnets such as neodymium-iron-boron (NdFeB), samarium cobalt, ferrite magnets, or other magnetic materials configured to provide static or semi-static magnetic fields. In some embodiments, electromagnets and permanent magnets may be used together to generate composite magnetic field profiles for manipulating microrobots.

The portable system combines all these necessary components for microrobot control into a single, compact unit within a system housing. The portable design may incorporate features such as a user-friendly control interface, allowing operators to input commands and monitor system performance through software running on an external computer. The system may communicate with the signal generator via a USB interface, enabling seamless integration with existing laboratory equipment. The system may also communicate with the signal generator using Ethernet, Bluetooth, serial connections, or other interfaces.

Advantages of the all-in-one portable design include increased mobility, allowing for microrobot experiments and applications in diverse settings beyond traditional laboratory environments. The integrated nature of the system may reduce setup time and complexity, potentially enabling faster deployment and more streamlined experimental protocols. By consolidating all necessary components into a single unit, the portable system may also improve reliability and reduce potential points of failure compared to setups using separate, discrete components. The compact nature of the integrated system may facilitate its use in space-constrained environments, such as clinical settings or field research locations. Additionally, the portability of the system may enable new applications for microrobot technology, such as on-site environmental monitoring, point-of-care medical diagnostics in austere environments, or rapid deployment for industrial inspection tasks. Additionally, the portable design allows the control system to be deployed within vehicles including cars, trucks, helicopters, airplanes, or vessels. As used herein, ‘portable’ refers to a system configured to be transported and operated outside of a fixed laboratory installation, including configurations that are self-contained within a single housing and operable using standard electrical power sources.

The integration of the power supply unit, signal generator, amplifier, coil assembly, and cooling system within a compact housing enables deployment in environments where conventional laboratory-scale systems are impractical, thereby expanding the range of applications for microrobot control systems.

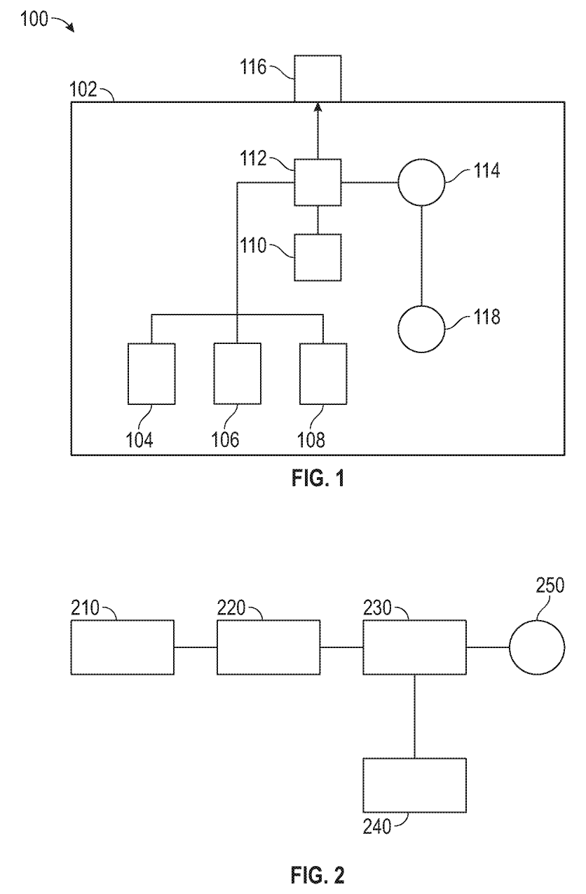

FIG. 1 illustrates an exemplary all-in-one magnetic control system. For example, the magnetic control system for microrobots may include a magnetic control system 100. In some cases, the magnetic control system 100 may comprise a system housing 102, which provides structural support, electromagnetic shielding, and thermal isolation for the internal components. The system housing 102 integrates all of the components described herein into a single integrated system.

The system housing 102 may be constructed from materials such as aluminum alloy, reinforced polymers, or composite materials selected for their non-magnetic properties, thermal conductivity, and mechanical strength. The system housing 102 may incorporate ventilation features strategically positioned to enhance airflow while maintaining electromagnetic shielding integrity. The system housing 102 may include access panels or removable sections to facilitate maintenance, component replacement, or system reconfiguration. In some embodiments, the system housing 102 may feature integrated mounting points for microscopes, cameras, or other observation equipment needed for microrobot experiments. The system housing 102 design may incorporate vibration-damping elements to isolate sensitive components from environmental disturbances. The exterior of the system housing 102 may include user interface elements such as status indicators, control buttons, or display screens for monitoring without requiring connection to an external computer. The dimensions and form factor of the system housing 102 may be optimized for portability while accommodating all necessary internal components, with potential variations including rack-mountable configurations for laboratory settings or ruggedized versions for field deployment. The magnetic control system 100 may include multiple elements for generating and controlling magnetic fields to manipulate microrobots with five degrees of freedom, enabling precise positioning and orientation control at the microscale.

The system housing 102 may include access panels or removable sections to facilitate maintenance, component replacement, or system reconfiguration. In some embodiments, the system housing 102 may feature integrated mounting points for microscopes, cameras, or other observation equipment needed for microrobot use. The system housing 102 design may incorporate vibration-damping elements to isolate sensitive components from environmental disturbances. The exterior of the system housing 102 may include user interface elements such as status indicators, control buttons, or display screens for system monitoring without requiring connection to an external computer. The dimensions and form factor of the system housing 102 may be optimized for portability while accommodating all necessary internal components, with potential variations including rack-mountable configurations for laboratory settings or ruggedized versions for field deployment.

The magnetic control system 100 includes multiple power supplies that form the foundation of the signal chain. The x-axis power supply 104, y-axis power supply 106, and z-axis power supply 108 collectively form a compact power supply unit that provides electrical power to multiple downstream components. The bipolar configuration of these power supplies enables bidirectional currents to be applied to the electromagnetic coils in the magnetic control assembly 114, allowing magnetic field generation in both positive and negative directions along each axis.

The power supplies may connect to a power distribution circuit before connecting to the amplifier 112, providing the necessary electrical power for signal amplification. The bipolar arrangement enables the amplifier 112 to produce both positive and negative output signals, which are essential for generating magnetic fields in multiple directions. This bidirectional capability allows the magnetic control assembly 114 to create complex field patterns by receiving varying current directions from the amplifier 112. The power supplies incorporate feedback circuits that monitor output parameters and communicate with both the amplifier 112 and the control interface 116, enabling real-time adjustment of power delivery based on system demands and user inputs. This feedback loop ensures that the power supplies maintain optimal voltage and current levels to the amplifier 112 as it drives the magnetic control assembly 114 under varying load conditions.

The signal generator 110 initiates the control signal path by generating precise waveforms that define the magnetic field patterns. These control signals pass through an intermediate conditioning and buffering gain stage before connecting to the amplifier 112, which processes and amplifies them while maintaining signal fidelity. The signal generator 110 receives instructions from the control interface 116, allowing users to specify desired magnetic field characteristics. Based on these inputs, the signal generator 110 produces appropriate waveforms—whether static, rotating, oscillating, or gradient fields—and sends these signals to the amplifier 112. The amplifier 112 then increases the available drive current and amplitude of these signals while preserving their waveform characteristics, transforming the low-power control signals from the signal generator 110 into high-power driving signals capable of energizing the electromagnetic coils in the magnetic control assembly 114. The amplifier 112 draws the necessary power from the x-axis power supply 104, y-axis power supply 106, and z-axis power supply 108, converting this electrical power into amplified control signals that match the waveform patterns specified by the signal generator 110.

The magnetic control assembly 114 receives the amplified signals from the amplifier 112 and emits the corresponding magnetic fields. As the amplified signals flow into the electromagnetic coils within the magnetic control assembly 114, they generate magnetic fields in the working space with an amplitude proportional to the commanded current. The strength, direction, and pattern of these magnetic fields directly correspond to the characteristics of the amplified signals received from the amplifier 112, which in turn reflect the original control signals generated by the signal generator 110 based on user inputs from the control interface 116. This signal chain—from user input through the control interface 116, to waveform generation in the signal generator 110, to power amplification in the amplifier 112, to field generation in the magnetic control assembly 114—creates a complete pathway for translating user commands into physical magnetic fields that manipulate the microrobots.

The magnetic control assembly 114 may incorporate one or more measuring devices that measure the phase currents of the electromagnetic coils of the magnetic control assembly 114. The phase currents of the coils are relayed back to the control interface 116, enabling closed-loop control. Since the properties of the coils are typically well-known, this current-based feedback approach ensures the generated fields match the intended specifications. Additionally, the magnetic control assembly 114 may include magnetic field sensors that measure the actual magnetic field parameters, primarily for fault detection purposes.

The control interface 116 serves as both the input and monitoring point in the system's signal flow. It receives user commands and translates them into specific control parameters for the signal generator 110, which then produces the appropriate waveforms. The control interface 116 also receives feedback from multiple system components—including power measurements from the power supplies, signal characteristics from the amplifier 112, field measurements from the magnetic control assembly 114, and temperature readings from the cooling system 118—creating a comprehensive monitoring and control hub. This bidirectional communication enables users to adjust parameters throughout the signal chain, from modifying the power output of the x-axis power supply 104, y-axis power supply 106, and z-axis power supply 108, to changing waveform characteristics in the signal generator 110, to fine-tuning amplification settings in the amplifier 112. The control interface 116 processes this information and displays it through visualization tools, allowing users to observe the relationship between their inputs and the resulting magnetic fields that control the microrobots. The control interface 116 may have remote operation capabilities, enabling control of the magnetic system from various locations. Users can access and operate the system through a computer connected to the same local area network (LAN) as the device, providing low-latency control within laboratory or industrial settings. Additionally, the system supports secure internet-based remote access, allowing authorized users to monitor experiments, adjust parameters, and control microrobot operations from any location with internet connectivity. This remote functionality is implemented through encrypted communication protocols that maintain data security while providing real-time control and feedback. The remote operation capabilities significantly enhance the system's utility for collaborative research, field deployments, and applications requiring operation in hazardous or inaccessible environments.

In some embodiments, feedback signals corresponding to coil current, voltage, or magnetic field measurements may be used in closed-loop control algorithms to dynamically adjust the generated magnetic fields. Such feedback-based control enables improved accuracy, stability, and repeatability of microrobot manipulation.

The cooling system 118 interacts thermally with multiple system components to maintain optimal operating temperatures. It forms a direct thermal connection with the magnetic control assembly 114, where it absorbs and dissipates the heat generated by the electromagnetic coils during operation. The cooling system 118 also provides thermal management for the amplifier 112, which produces significant heat while amplifying signals, and for the power supplies, which generate heat during power conversion. This thermal management system creates a closed-loop cooling pathway where heat is transferred from the active components to the cooling system 118, dissipating this thermal energy through its heat exchanger. The cooling system 118 communicates with the control interface 116, providing temperature data from various system components and receiving control commands that adjust cooling parameters such as pump speed and fan operation. This thermal feedback loop may ensure that all components in the signal chain—from the power supplies, the signal generator 110, the amplifier 112, and the magnetic control assembly 114—maintain temperatures within their optimal operating ranges, preserving system performance and reliability.

The cooling system 118 operates in coordination with a power management system, adjusting its cooling capacity based on the power levels being delivered by the power supplies to the amplifier 112 and ultimately to the magnetic control assembly 114. When the system operates at higher power levels, such as when generating stronger magnetic fields or higher frequency field rotations, the cooling system 118 increases its cooling capacity to manage the additional heat generated throughout the signal chain. The cooling system 118 receives operational data from the signal generator 110 regarding the waveform patterns being produced, allowing it to anticipate thermal loads based on the specific magnetic field configurations being generated. In one embodiment, when the signal generator 110 produces high-frequency rotating field patterns that will cause the amplifier 112 to drive the magnetic control assembly 114 at higher power levels, the cooling system 118 proactively increases coolant flow to these components. This integrated approach to thermal management ensures that the entire signal chain—from power input through signal generation, amplification, and field production—maintains optimal operating conditions regardless of the specific magnetic field patterns being generated. The system housing integrates all these components and their interactions into a portable package, enabling the complete signal chain from power input to magnetic field output to function cohesively in field deployment scenarios.

The cooling system 118 may include a water pump and a cold plate in addition to the water blocks. The water pump circulates coolant through the channels and other components, with variable speed control to adjust cooling capacity based on thermal load. The cold plate serves as a heat exchanger, incorporating fins or other extended surfaces to maximize heat transfer to the ambient air, and may be actively cooled with fans. The system may include a reservoir to facilitate air bleeding and provide connection points for expanding the cooling loop, with temperature sensors at various points to monitor performance and provide feedback for control algorithms.

In one embodiment, the magnetic control system 100 may be configured for a method of microplastic extraction from aquatic environments. In this embodiment, the system controls microrobots specifically designed to capture and remove microplastic particles from water. These microrobots incorporate magnetic materials as described previously but also feature specialized surface functionalization to interact with microplastics. The microrobots may employ one or more mechanisms for microplastic capture, including hydrophobic surface domains, mechanical structures, or chemical binding sites. For example, the microrobots may be fabricated with regions coated with hydrophobic polymers such as fluorinated compounds that preferentially attract and bind to the predominantly hydrophobic surfaces of microplastic particles. These hydrophobic domains may be patterned in specific geometries to maximize contact area with microplastics while maintaining the microrobot's mobility. Alternatively, the microrobots may incorporate physical capture mechanisms such as micro-hooks, barbs, or adhesive regions that mechanically entrap microplastic particles between 20-500 μm in size. Some implementations may utilize microrobots with surface-bound antibodies, coatings, or synthetic receptors that specifically recognize common polymer components found in microplastics, such as polyethylene, polypropylene, or polyethylene terephthalate. Alternatively, electrostatic coatings may be used to attract microplastics to microrobot surfaces. In further embodiments, microrobots may include surfaces comprised of polymers that may have hydrophobic portions and/or hydrophilic portions.

In alternative embodiments, the cooling system 118 may include air-based cooling, passive heat sinks, thermoelectric cooling devices, phase-change cooling systems, or immersion cooling techniques. The cooling system 118 may be implemented in open-loop or closed-loop configurations and may utilize a variety of coolant fluids or thermal transfer media.

The magnetic control system 100 enables precise navigation of these functionalized microrobots through contaminated water. The signal generator 110 may be programmed with specific waveform patterns that create sweeping or spiral motion paths, maximizing the water volume covered by the microrobots. The magnetic control assembly 114 generates field gradients that guide the microrobots through predetermined collection routes, with field strengths modulated between 2-8 mT to optimize microrobot-microplastic interaction. For example, in a typical extraction operation, contaminated water may be introduced into the working space 308 of the coil assembly 300. The microrobots are then deployed and controlled using rotating magnetic fields generated by the x-axis coils 302, y-axis coils 304, and z-axis coils 306. As the microrobots navigate through the water, their functionalized surfaces capture microplastic particles. The system may employ a multi-stage approach where microrobots first perform broad sweeping patterns at higher speeds (using 5-10 Hz rotating fields) to cover large volumes, followed by slower, more localized movements (using 1-3 Hz fields) in areas with higher microplastic concentrations. Once the microrobots have collected sufficient microplastics, the magnetic control assembly 114 generates stronger localized magnetic fields (10-15 mT) to direct the microrobots to designated collection zones. These collection zones may feature stronger field gradients (0.3-0.5 T/m) that concentrate the microrobots and their captured microplastic cargo. The microplastics can then be removed from the system through mechanical harvesting, sonication, filtration, or fluid exchange while the microrobots are held in place magnetically. Alternatively, the microrobots with microplastics may be collected and removed from the system entirely. If reusing microrobots is desired, after microplastic removal from the microrobots, the microrobots can be redirected back into the treatment volume for additional collection cycles.

In one embodiment for environmental remediation, the system may be configured with a continuous-flow that processes 1-5 liters of water per hour. The microrobots, measuring 100-300 μm in diameter, feature a core-shell structure with magnetic nanoparticles (Fe₃O₄, 10-50 nm) embedded in a biodegradable polymer matrix core, surrounded by a functionalized shell. The shell combines hydrophobic PDMS regions with microstructured physical entrapment features that can capture microplastics ranging from 20-500 μm. The control interface 116 provides real-time monitoring of microplastic concentration through integrated optical sensors, with automated adjustment of magnetic field parameters to optimize collection efficiency. The system maintains microrobot swarms in continuous operation for up to 8 hours before requiring regeneration or replacement, achieving microplastic removal efficiencies of 75-90% depending on particle size and concentration. This embodiment demonstrates how the portable magnetic control system enables practical environmental applications beyond traditional laboratory settings, addressing critical environmental challenges through precise microrobot manipulation.

A method of operating the magnetic control system 100 for microplastic extraction comprises: providing electrical power through the power supply unit including the x-axis power supply 104, y-axis power supply 106, and z-axis power supply 108; generating control signals using the signal generator 110, wherein the control signals define magnetic field patterns for guiding microrobots through contaminated water; amplifying the control signals using the amplifier 112, wherein the amplifier 112 draws power from the power supplies to transform low-power control signals into high-power driving signals; transmitting the amplified signals to the magnetic control assembly 114, wherein the magnetic control assembly 114 converts the amplified signals into controlled magnetic fields within a working space; dissipating heat generated by the magnetic control assembly 114 using the cooling system 118, wherein the cooling system 118 maintains optimal operating temperatures throughout the system; receiving user inputs through the control interface 116, wherein the control interface 116 allows operators to specify desired magnetic field characteristics; processing feedback from multiple system components through the control interface 116, including power measurements from the power supplies, signal characteristics from the amplifier 112, field measurements from the magnetic control assembly 114, and temperature readings from the cooling system 118; and adjusting control parameters based on the feedback to optimize microrobot navigation and microplastic collection efficiency.

FIG. 2 illustrates a detailed view of the signal generation and amplification components of the magnetic control system 100. The figure depicts a controller 210, which serves as a component of the signal generator 110 shown in FIG. 1. In some embodiments, the controller 210 comprises a microcontroller capable of generating high-fidelity signals up to kilohertz frequencies with 8-bit resolution. The controller 210 comprises a microcontroller capable of generating high-fidelity signals up to kilohertz frequencies with 12-bit resolution, or 16-bit resolution, or 24-bit resolution. This precision enables accurate control of magnetic field parameters for microrobot manipulation. The controller 210 may be based on a microcontroller such as a Raspberry Pi Pico, ARM Cortex-M series, or similar embedded processor. The controller 210 incorporates a high-speed processor core operating at frequencies between 48 MHz and 133 MHz, with dedicated hardware peripherals for precise timing control and waveform generation. The microcontroller includes onboard flash memory ranging from 256 KB to 2 MB for storing waveform patterns and control algorithms, along with 32 KB to 264 KB of SRAM for runtime operations. This controller 210 provides the high-fidelity signal generation capabilities referenced in FIG. 1, but with additional details about its 12-bit resolution and kilohertz frequency capabilities that were not specified in the system-level diagram.

Connected to the controller 210 is a resistor ladder 220, which is a subcomponent of the signal generator 110 from FIG. 1. The resistor ladder 220 functions as a digital-to-analog converter (DAC) that transforms the digital control signals from the controller 210 into analog waveforms. The resistor ladder 220 component represents how the signal generator 110 in FIG. 1 converts digital representations of desired magnetic field patterns into the analog signals needed to drive the amplifier and coil assembly.

The resistor ladder 220 receives digital bit patterns from the controller 210 and converts them into corresponding voltage levels using a network of resistors with ±0.1% tolerance. Each digital bit controls a specific weighted current path in the resistor network, with the sum of these currents creating a proportional output voltage. Operational amplifiers with input offset voltages below 100μV and open-loop gains exceeding 100dB buffer the voltage references, and provide a low-impedance output stage. This architecture enables the resistor ladder 220 to generate analog signals with up to 12-bit resolution (4096 discrete voltage levels) and settling times under 1μs.

To maintain signal fidelity across operating conditions, the resistor ladder 220 incorporates temperature compensation techniques, including resistors with low temperature coefficients (less than 25 ppm/°C) and PCB layouts that minimize thermal gradients. This ensures consistent performance across the operating temperature range of 0°C to 70°C. After digital-to-analog conversion, the signals undergo low-pass filtering with cutoff frequencies between 100 kHz and 1 MHz to remove high-frequency noise and quantization artifacts while preserving the essential waveform characteristics. The resulting high-fidelity analog signals are then sent to the linear amplifier circuit 230 for power amplification before driving the magnetic control assembly.

The linear amplifier circuit 230 shown in FIG. 2 corresponds directly to the amplifier 112 block in FIG. 1. The linear amplifier circuit 230 comprises a bridge-tied load configuration that takes the analog signals from the resistor ladder 220. This configuration utilizes two amplifier channels operating in a differential mode, where one amplifier drives the positive signal while the other drives an inverted version of the same signal. The bridge-tied load arrangement doubles the effective voltage swing across the load compared to single-ended configurations, enabling higher power delivery to the electromagnetic coils without increasing supply voltage. The linear amplifier circuit 230 incorporates high-current output stages with discrete power transistors arranged in complementary pairs (NPN/PNP or N-channel/P-channel), typically mounted on aluminum heatsinks with thermal interface materials to facilitate heat dissipation. The linear amplifier circuit 230 includes feedback networks with resistors (±0.1% tolerance) that establish gain characteristics while maintaining stability across the operating bandwidth. Protection circuitry within the amplifier prevents damage from output shorts, thermal overload, or excessive current draw, with fault detection systems that can disable output stages and signal the control system when abnormal conditions are detected. For applications requiring higher frequency operation, the linear amplifier circuit 230 may be configured with reduced feedback capacitance and optimized compensation networks to extend bandwidth to 20-30 kHz, though with potentially increased distortion characteristics of 0.2-0.3%. Alternatively, for applications prioritizing precision over bandwidth, the linear amplifier circuit 230 may be configured with feedback networks and components to achieve ultra-low distortion (0.01-0.05% THD) over a more limited bandwidth of DC to 5 kHz. For high-power applications, the amplifier may incorporate parallel output stages with current-sharing networks to distribute thermal load across multiple transistor pairs.

In alternative embodiments, the signal generator 110 may include other waveform generation architectures, including field-programmable gate arrays (FPGAs), direct digital synthesis (DDS) modules, analog waveform generators, or hybrid digital-analog signal generation systems. The signal generator 110 may also incorporate external digital-to-analog converters, integrated DAC peripherals, or software-defined waveform generation systems.

FIG. 2 further illustrates the power supply 240 that energizes the linear amplifier circuit 230. The power supply 240 corresponds to the collection of power supplies shown in FIG. 1, specifically the x-axis power supply 104, y-axis power supply 106, and z-axis power supply 108. While FIG. 1 shows these as three separate power supply blocks feeding into the amplifier 112, FIG. 2 simplifies this representation into a single power supply 240 block to focus on the signal path rather than the power distribution architecture. The power supply 240 in FIG. 2 provides additional technical details about the power conditioning features (switched-mode design, power factor correction, filtering and regulation stages) that were not visible in the system-level power supply blocks of FIG. 1, highlighting how the power supplies support the amplifier's operation.

While linear amplifiers are described in certain embodiments, other amplification architectures may be employed, including switching amplifiers such as Class D amplifiers, hybrid amplifier configurations, or resonant drive circuits, depending on efficiency, bandwidth, and thermal requirements.

The magnetic control assembly 250 shown in FIG. 2 directly corresponds to the magnetic control assembly 114 depicted in FIG. 1. Both representations serve the same function in their respective diagrams—receiving amplified signals and generating controlled magnetic fields. However, FIG. 2 provides enhanced technical details about the magnetic control assembly's implementation that were not visible in FIG. 1, including the Helmholtz or Maxwell coil configuration, the use of high-temperature-resistant wire and ferromagnetic cores, and specific performance parameters such as field switching rise times. These details expand upon the basic functional representation shown in FIG. 1, providing insight into how the magnetic control assembly 114 achieves the magnetic field generation capabilities referenced in the system overview.

As depicted in FIG. 2, the signal path flows sequentially from the controller 210 through the resistor ladder 220 and linear amplifier circuit 230 to the magnetic control assembly 250, with the power supply 240 providing the necessary energy for amplification. This signal chain directly corresponds to the flow shown in FIG. 1 from the signal generator 110 through the amplifier 112 to the magnetic control assembly 114, powered by the x-axis power supply 104, y-axis power supply 106, and z-axis power supply 108. The primary difference is that FIG. 2 expands upon the internal components and connections that were simplified or abstracted in the system-level diagram of FIG. 1. Notably, FIG. 2 does not explicitly show the control interface 116 or cooling system 118 that appear in FIG. 1, as FIG. 2 focuses specifically on the signal generation and amplification subsystems rather than the complete system architecture. The complete signal generation and amplification system shown in FIG. 2 provides a more detailed technical view of the signal generator 110 and amplifier 112 components referenced in FIG. 1, revealing the implementation details that enable the magnetic control system's operation.

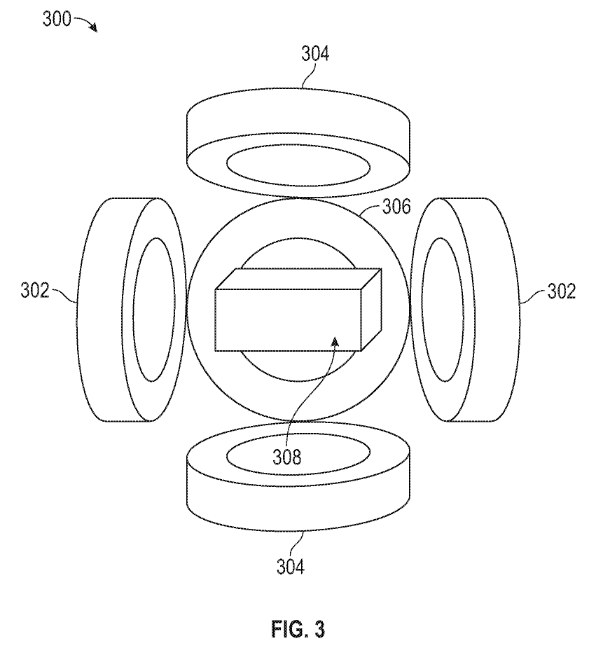

FIG. 3 illustrates an exploded view of the coil assembly 300. The magnetic control system 100 may include a coil assembly 300 for generating magnetic fields to control microrobots. The coil assembly 300 forms a part of the magnetic control assembly 114, as illustrated in FIG. 1, enabling the generation and modulation of magnetic fields based on the amplified signals received from the amplifier 112. This integration allows the magnetic control assembly 114 to convert electrical signals into the controlled magnetic fields necessary for microrobot manipulation within the working space. FIG. 3 provides a visualization of the spatial arrangement of electromagnetic coils that form the core of the magnetic field generation system. The exploded view reveals how the coils are positioned relative to each other to create a three-dimensional control volume for microrobot manipulation. The coil assembly 300 may include three pairs of electromagnetic coils arranged orthogonally to each other, enabling independent control of magnetic fields along the x, y, and z axes.

The coil assembly 300 may include two x-axis coils 302 positioned on opposite sides of the assembly. Two y-axis coils 304 may be positioned above and below the assembly. Two z-axis coils 306 may be positioned on the remaining opposing sides. The orthogonal arrangement of coil pairs is integrated into the magnetic control assembly 114 shown in FIG. 1, where it receives amplified signals from the amplifier 112 to generate controlled magnetic fields. The coil pairs connect to the linear amplifier circuit 230 depicted in FIG. 2, which provides the necessary power amplification through the bridge-tied load configuration. Each coil pair may be independently controlled by separate channels of the amplifier 112, with control signals originating from the signal generator 110 and processed through the controller 210 and resistor ladder 220 shown in FIG. 2. The orthogonal arrangement of the coil pairs may enable vector summation of the individual axis fields to create resultant fields in any desired direction within the working space. This configuration may also allow for the generation of rotating magnetic fields by applying time-varying sinusoidal currents with appropriate phase relationships to the different coil pairs, with these complex waveforms being generated by the controller 210 and converted to analog signals by the resistor ladder 220 before amplification.

The x-axis coils 302, y-axis coils 304, and z-axis coils 306 may be arranged to create a working space 308 at the center of the coil assembly 300. The working space 308 may be defined by the overlapping regions between the three coil pairs. In some embodiments, the working space 308 size may vary depending on the power supplied to the coils. In some cases, the working space 308 may have dimensions of 2 cm x 2 cm x 2 cm, providing a volume for controlling microrobots. For applications requiring larger manipulation areas, the working space may be expanded to dimensions of 3 cm x 3 cm x 3 cm or 4 cm x 4 cm x 4 cm, with corresponding volumes of 27 cm³ and 64 cm³, respectively. In some embodiments, the working space 308 may be larger than described herein. For more precise micromanipulation tasks, a smaller working space of 1 cm x 1 cm x 1 cm may be configured to achieve higher field strengths. Within this working space, the magnetic field may maintain uniformity within ±5% of the target value, ensuring consistent control forces on the microrobots regardless of their position. The working space 308 may be designed to be accessible for observation and manipulation, with sufficient clearance for microscopes, cameras, or other instruments needed for microrobot experiments.

The working space 308 may also function as a sample holder, incorporating features such as a transparent bottom surface for imaging, removable side walls for sample access, and fluid ports for media exchange. The sample holder configuration may include recessed wells with depths ranging from 0.5 mm to 5 mm to contain liquid samples while allowing microrobot navigation. For microplastic extraction applications, contaminated fluid may be introduced through an inlet port at flow rates between 0.1-5 mL/min, with the flow pattern controlled by micropillars or channel structures to create laminar flow regions and vortex zones that enhance microplastic separation. The working space may incorporate a staged filtration system with decreasing pore sizes (500 μm to 10 μm) to capture microplastics of various dimensions while allowing the microrobots to navigate freely through dedicated bypass channels of 600-800 μm width. Magnetic field gradients generated by the coil assembly 300 may guide the microrobots in sweeping patterns through the contaminated material, with field strengths modulated between 2-8 mT to optimize microrobot-microplastic interaction. The microrobots may employ surface functionalization with hydrophobic domains that preferentially bind to microplastic particles, or mechanical structures such as micro-hooks or adhesive regions that physically capture particles between 20-500 μm in size. Collection zones within the working space may feature stronger localized magnetic fields (10-15 mT) to concentrate microrobot-captured plastics, with automated extraction mechanisms such as secondary flow channels, mechanical harvesting systems, or magnetic removal to remove accumulated microplastics while returning cleaned microrobots to the active filtration region.

Each of the x-axis coils 302, y-axis coils 304, and z-axis coils 306 may be constructed using materials and configurations to achieve desired magnetic field characteristics. In some cases, the coils may use 26 AWG wire, 24 AWG wire, 28 AWG wire, or 30 AWG wire depending on current requirements and space constraints. The coils may be constructed from various conductive materials including oxygen-free high-conductivity (OFHC) copper wire with 99.99% purity, silver-plated copper wire for reduced skin effect at higher frequencies, aluminum wire for weight-sensitive applications, or copper-clad aluminum wire offering a balance between conductivity and weight. For specialized high-performance applications, the coils may utilize litz wire constructed from multiple insulated strands to reduce losses at higher frequencies. Each coil may have 600 turns of wire to generate a magnetic field strength. Alternative configurations may include 300-500 turns for applications requiring faster field changes, 700-900 turns for higher field strength applications, or 100-200 turns for low-inductance, rapid-response scenarios. For specialized gradient coil applications, the system may employ 1000-1500 turns to generate stronger field gradients. The wire may be insulated with high-temperature polyimide coating to withstand the heat generated during operation. Other insulation options may include modified polyester for general-purpose applications, polyurethane-nylon composite for excellent mechanical durability, polyester-imide for good thermal performance up to 180°C, PTFE (polytetrafluoroethylene) coating for chemical resistance and high-temperature stability up to 250°C, or silicone-based insulation for applications requiring flexibility and thermal cycling resistance. For extreme temperature applications, ceramic-based insulation systems capable of withstanding temperatures up to 500°C may be employed. The coils may be wound on non-conductive, non-magnetic formers to maintain their shape and provide structural support. In some embodiments, the coils may incorporate ferromagnetic cores or pole pieces to enhance field strength and shape the field distribution. The coil windings may be secured with high-temperature epoxy to prevent movement or deformation during thermal cycling.

The arrangement of the x-axis coils 302, y-axis coils 304, and z-axis coils 306 may allow for control of magnetic fields within the working space 308. By independently controlling the current through each coil pair, the magnetic control system 100 may generate magnetic fields of varying strength and direction. This configuration may enable complex manipulation of microrobots within the working space 308. The system may be capable of generating static fields for holding microrobots in position, rotating fields for inducing spinning or swimming motions, oscillating fields for vibrational actuation, and field gradients for applying directional forces. The independent control of each axis may also enable the implementation of closed-loop control algorithms that adjust field parameters based on feedback from imaging systems tracking microrobot positions.

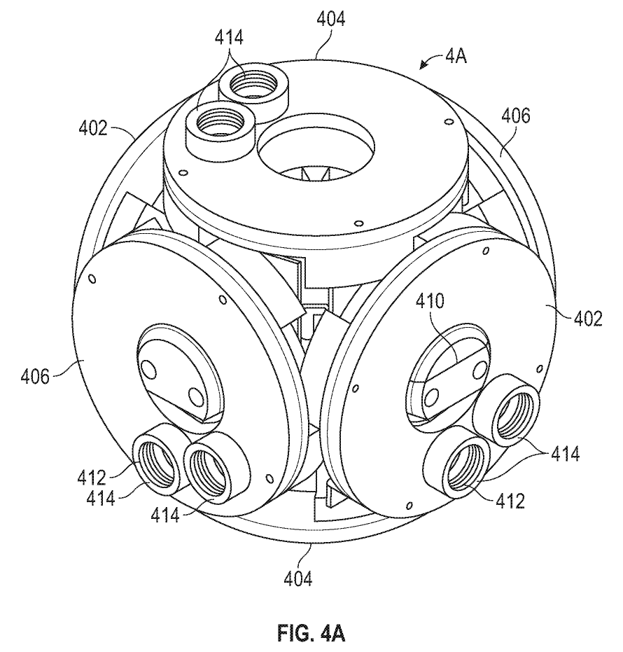

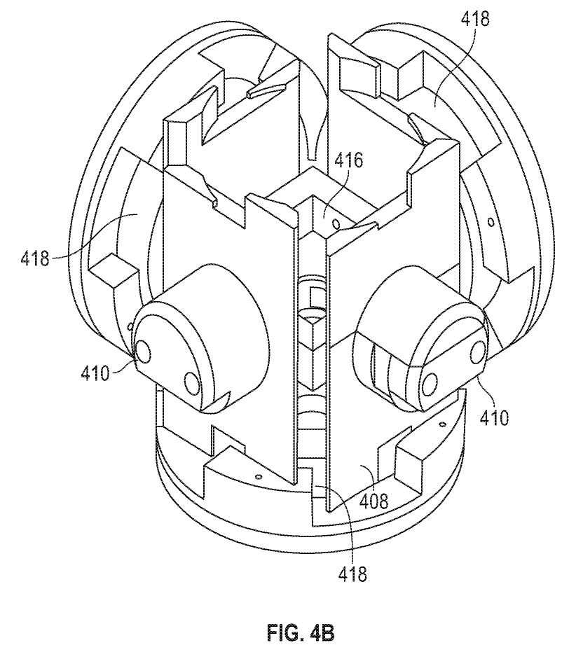

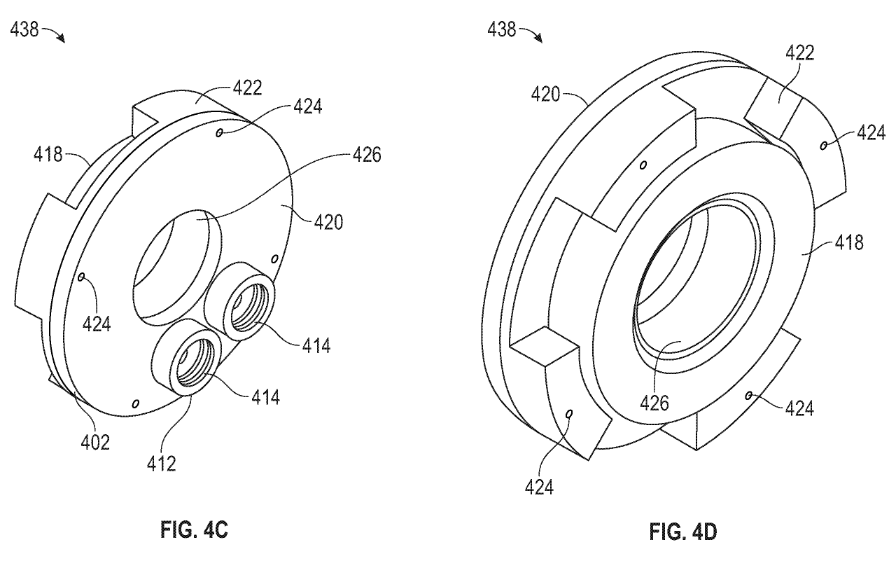

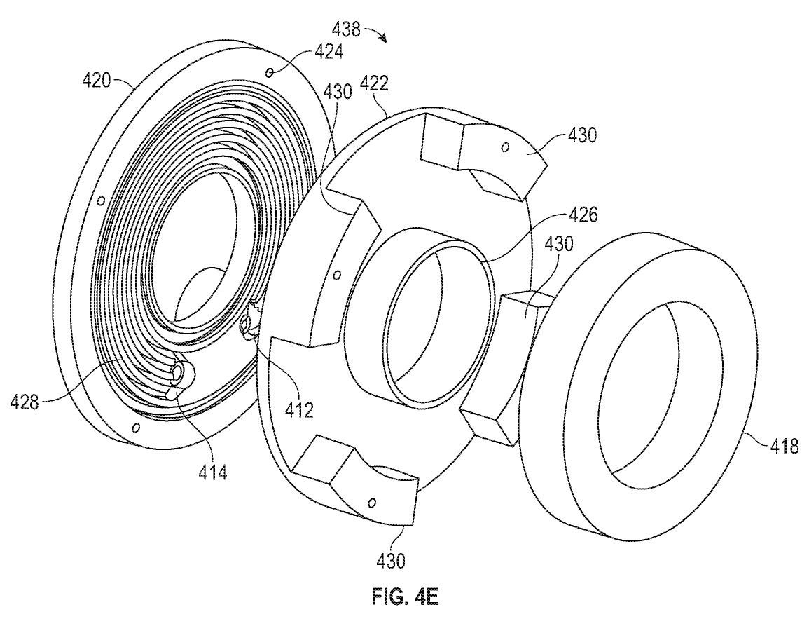

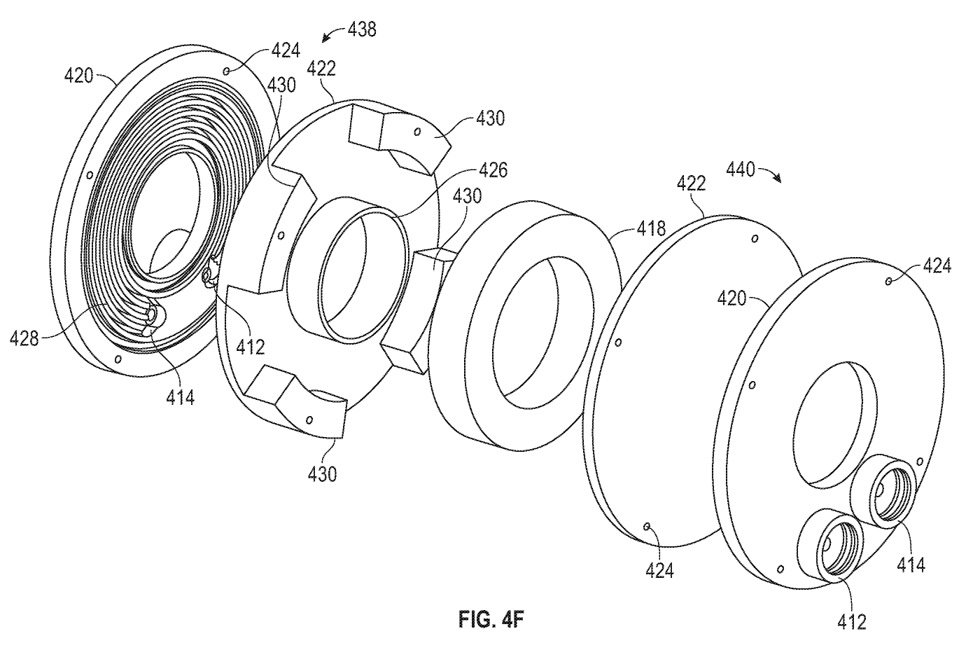

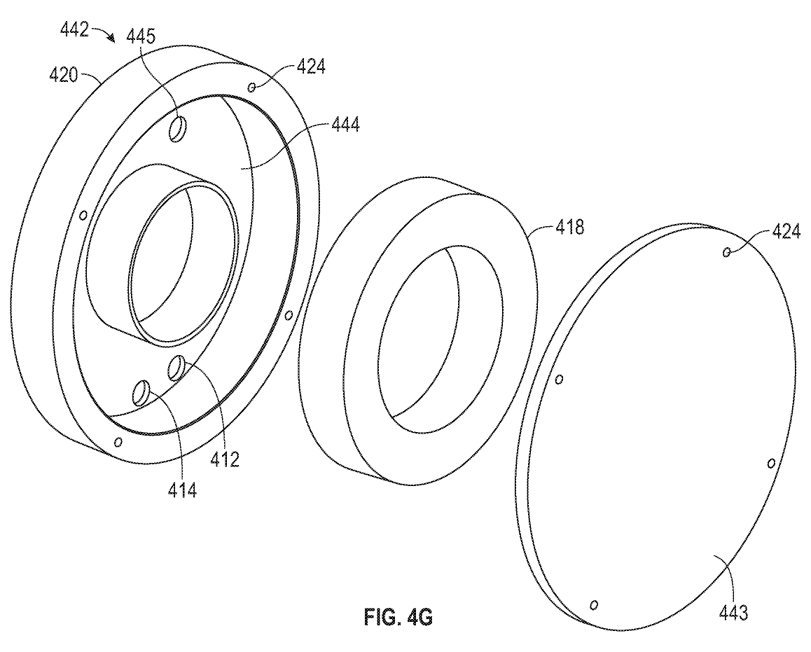

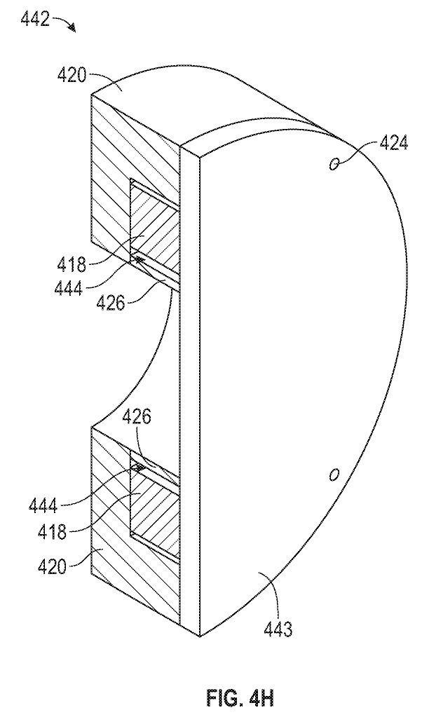

FIG. 4A illustrates an isometric view of a magnetic control assembly 400, such as the magnetic control assembly 114 described in FIG. 1. The isometric view provides a three-dimensional perspective of the complete assembly, showing the spatial arrangement and integration of various components. The magnetic control assembly forms a component of the magnetic control assembly 114 shown in FIG. 1, where it receives control signals from the amplifier 112 to generate the precise magnetic fields required for microrobot manipulation. The magnetic control assembly 114 includes the coil assembly 300 and associated structural, sensing, and cooling interface components. The magnetic control assembly 114 may include multiple components arranged to create a spherical working space for magnetic field generation, with integrated cooling systems to maintain thermal stability during operation. The magnetic control assembly 114 may include an x-axis coil mount 402, a y-axis coil mount 404, and a z-axis coil mount 406 arranged orthogonally to enable magnetic field generation along different axes. These coil mounts may be configured to hold electromagnetic coils that form part of the coil assembly 300. Each coil mount may be machined from non-magnetic materials such as aluminum or polymer composites to ensure dimensional accuracy and stability, with features for precise positioning and alignment of the coils. The design may include provisions for thermal expansion during operation while maintaining critical geometric relationships between coils.

Positioning components 410 may help align and secure the coil mounts in their proper orientations, ensuring precise alignment to maintain proper magnetic field generation capabilities within the working space 308. These components may include machined brackets, alignment pins, or adjustable fixtures that enable fine-tuning of the coil positions during assembly and calibration. The positioning components 410 may be designed to maintain alignment stability under thermal cycling and mechanical vibration, ensuring consistent magnetic field generation over extended operating periods.

The magnetic control assembly may include a coolant inlet 412 and a coolant outlet 414 integrated into the coil mounts to enable liquid cooling of the electromagnetic coils during operation. The coolant connections may utilize quick-disconnect fittings to facilitate assembly and maintenance while preventing leakage. The coolant flow paths may be designed to maximize thermal transfer efficiency, with turbulence-inducing features to enhance heat exchange between the coils and the coolant. The cooling system 118 may be capable of dissipating up to 1000W of heat while maintaining coil temperatures below 60°C.

FIG. 4B illustrates a section view of the magnetic control assembly, such as the magnetic control assembly 114 described in FIG. 1. The cross-sectional view shows the spatial relationships between the support structure, coils, and working space, highlighting the design considerations for achieving effective magnetic field generation while maintaining structural integrity. The magnetic control assembly may include a mounting block 408 that provides a secure foundation for attaching other components. The mounting block 408 may be machined from non-magnetic materials and incorporate features such as threaded holes, alignment pins, or keyed interfaces to facilitate precise assembly and alignment of the coils and support structures. The mounting block 408 may also include provisions for thermal management, such as integrated coolant channels or mounting points for temperature sensors.

The positioning components 410 may be arranged on the inner surfaces of each of the x-axis coil mount 402, the y-axis coil mount 404, or the z-axis coil mount 406 to ensure proper alignment and secure mounting as each coil mount slides onto and engages with its corresponding positioning component. The positioning components 410 may be designed to fit inside the x-axis coil mount 402, the y-axis coil mount 404, or the z-axis coil mount 406 in a piston-like arrangement, allowing for precise alignment and secure mounting while providing adjustability. For example, the positioning components 410 may be inserted into the positioning sleeve 426 of any of the x-axis coil mount 402, the y-axis coil mount 404, or the z-axis coil mount. The inner surfaces of the coil mounts may feature cylindrical bores, such as the positioning sleeve 426 that closely match the outer dimensions of the positioning components 410, creating a snug, sliding fit like a piston in a cylinder. This arrangement allows for smooth linear motion during assembly while maintaining tight tolerances for accurate positioning. The piston-like fit may incorporate features to enhance stability and prevent unwanted rotation, such as longitudinal keys that mate with corresponding keyways in the coil mount bores.

In some implementations, the piston-like fit may include provisions for fine-tuning the position of the coils, such as threaded sections or adjustable stops that allow for precise control over the insertion depth into the coil mounts. The surfaces of both the positioning components and the coil mount bores may be machined to tight tolerances and may receive surface treatments to reduce friction and wear. The positioning components 410 may include internal locking mechanisms that engage when the coil mount is fully seated, with materials selected to create an optimal sliding interface while matching the thermal expansion characteristics of the coils and support structures.

A working space 416 may be created at the center of the magnetic coil assembly, defining the volume where magnetic fields can be generated and controlled for microrobot manipulation. This working space may correspond to the working space 308 described in relation to the coil assembly 300. For micromanipulation tasks, the working space may have dimensions of 1 cm × 1 cm × 1 cm, while larger applications may utilize expanded dimensions of 3-4 cm in each direction. Within this working space, the magnetic field may maintain uniformity within ±5% of the target value, ensuring consistent control forces regardless of microrobot position. The working space 416 may be configured in various ways, including as a sealed volume for controlled environment experiments, a sample holder with one or multiple wells, an open container for easy access, a flow-through chamber for continuous media exchange, or a microfluidic channel for precise fluid control. For microplastic extraction applications, it may feature recessed wells with depths ranging from 0.5 mm to 5 mm. The boundaries may be defined by non-magnetic, optically transparent materials such as borosilicate glass or polycarbonate to facilitate observation while maintaining structural integrity.

The magnetic control assembly may include a coil 418, like the x-axis coil 302 described previously. Multiple instances of the coil 418 may be arranged symmetrically around the working space 416 to enable controlled magnetic field generation along different axes. The section view in FIG. 4B shows how the mounting block 408 and positioning components 410 are configured to hold the coil 418 in precise alignment while maintaining access to the working space 416, with the positioning components 410 interfacing with the mounting block 408 to create a stable assembly. The magnetic control assembly may include provisions for electrical connections, with terminal blocks or connectors positioned for easy access during assembly and maintenance. The design may incorporate features for signal integrity and include provisions for monitoring and feedback, such as mounting points for temperature sensors or Hall effect sensors for magnetic field measurement.

FIGS. 4C through FIG. 4D illustrate isometric views of the water-cooled coil assembly of the magnetic control assembly, such as the magnetic control assembly 114 described in FIG. 1. These figures reveal the external features of the cooling system, including coolant connections and mounting provisions, highlighting the compact and integrated nature of the design that enables sustained high-power operation without thermal degradation. The water-cooled coil assemblies disclosed herein are not limited to being cooled by water and may be cooled by any suitable coolant fluid in accordance with the embodiments disclosed herein. Further, the water-cooled coil assemblies disclosed below in FIGS. 4C through FIG. 4H may house and include a respective coil mount. For example, the water-cooled coil assemblies may be configured to house and extract heat from any or all the coil 418 instances arranged symmetrically around the working space 416 (e.g., coil 418 instance of x-axis coil mount 402, the y-axis coil mount 404, and/or the z-axis coil mount 406).

For example, the water-cooled coil assembly may include the x-axis coil mount 402 configured to house the coil 418. The x-axis coil mount 402 may be designed with specific features to support the coil while facilitating efficient cooling, with machined surfaces to ensure proper positioning and alignment. The material selection may balance thermal conductivity for heat transfer with non-magnetic properties to prevent interference with the generated fields.

The water-cooled coil assembly may include a first water block 438 comprising an outer water block portion 420 and an inner water block portion 422 that together form a housing for the x-axis coil mount 402 or y-axis coil mount 404 or z-axis coil mount 406. The inner water block portion 422 serves as the primary thermal interface, directly contacting the coil windings to maximize heat extraction. It may be fabricated from high thermal conductivity materials such as copper (approximately 400 W/m·K) or aluminum alloys (150-220 W/m·K) with a precisely machined inner diameter matching the coil's outer diameter. The inner surface may incorporate micro-texturing or grooves to increase the effective contact area, enhancing thermal transfer efficiency by up to 15-20%. In some implementations, it may be coated with a thin layer (0.05-0.2mm) of thermally conductive but electrically insulating material such as aluminum oxide or boron nitride.

The outer water block portion 420 forms the external shell of the cooling assembly and may be constructed from the same material as the inner block for thermal expansion compatibility or from different materials optimized for specific functions. It may feature external mounting provisions with 4-8 fastening points distributed evenly around its circumference. The outer diameter may range from 1.5-2.5 times the coil diameter, balancing compact design with sufficient coolant volume. In advanced implementations, it may include integrated temperature sensor mounting points with standardized M3 or M4 threaded holes.

Coolant channels 428 are engraved or embedded in the outer water block portion 420 of the first water block 438, as illustrated in FIG. 4E. The coolant channels 428 are positioned such that they allow for coolant to flow when the outer water block portion 420 and the inner water block portion 422 are secured together. The coolant channels 428 form a network of flow paths designed to maximize cooling efficiency while maintaining uniform temperature distribution. The coolant channels may be implemented in various configurations, including spiral, parallel, or helical geometries, with typical dimensions ranging from 2-5mm in width and 3-8mm in depth, creating a total coolant volume of 15-40ml within each assembly. The coolant channels 428 may incorporate flow directors or turbulators, small protrusions extending 0.5-1.5mm into the channel, positioned at 10-15mm intervals to enhance heat transfer by creating controlled turbulence. The coolant channel 428 walls may feature micro-fin structures with heights of 0.3-0.8mm to increase the effective surface area for heat exchange by 30-50%.

The coolant inlet 412 and coolant outlet 414 enable a complete thermal management cycle where coolant enters at temperatures typically between 15-25°C, absorbs heat as it traverses the channels, and exits at temperatures 5-15°C higher, depending on flow rate and heat load. The flow follows an engineered path to ensure all regions receive adequate cooling, with flow rates that may range from 0.5-2.0 liters per minute, from 0.1-3.5 liters per minute, from 1-8 liters per minute, per assembly. Other flow rates may be contemplated within the ranges described above. The inlet and outlet connections may be positioned to facilitate natural air bleeding during system filling, with the inlet typically located lower than the outlet. Depending on specific thermal requirements and pressure drop considerations, the coolant flow may be arranged in parallel or series configurations across multiple assemblies.

A positioning sleeve 426 integrated into the water-cooled coil assembly on the inner water block portion 422 helps maintain proper alignment of the components. The coil 418 is seated on the outer portion of the positioning sleeve 426, creating a concentric arrangement where the cylindrical outer surface provides a machined mounting surface. This seating arrangement establishes a fixed radial position relative to the central axis, ensuring consistent magnetic field generation. The outer portion includes a raised annular ridge that engages with a corresponding channel in the inner diameter of the coil 418, preventing axial movement while allowing for thermal expansion. The inner portion of the positioning sleeve 426 interfaces with the positioning components 410. When assembled, these interlocking features create a connection that establishes and maintains the orthogonal relationship between different coil assemblies. The inner portion of the positioning sleeve 426 may also include keyed channels (not shown) that engage with complementary features on the positioning components 410, preventing rotational movement while allowing for controlled thermal expansion.

Multiple fastening holes 424 arranged around the periphery of the water block portions secure the assembly together, ensuring proper alignment and sealing of the coolant channels. These holes accommodate high-strength fasteners that maintain consistent clamping force across thermal cycles, with the pattern arranged to provide uniform compression of sealing elements. The number and spacing of fasteners are determined based on mechanical analysis to ensure adequate sealing pressure while preventing distortion, and the fastening system includes provisions for torque specification and locking to prevent loosening during operation.

Positioning blocks 430 and positioning sleeve 426 help maintain proper alignment of the components when assembled. The positioning blocks 430 feature a piston-like geometry with protrusions that interlock with corresponding receptacles in adjacent coil mounts, creating a secure mechanical connection between orthogonal assemblies. This interlocking mechanism includes machined joints along the edges where positioning blocks 430 meet, which slide together and lock when fully seated, preventing lateral movement while allowing for controlled thermal expansion. The positioning blocks 430 are placed at the intersection points of the x-axis coil mount 402, y-axis coil mount 404, and z-axis coil mount 406, where they serve as structural nodes that distribute mechanical forces evenly throughout the assembly. Different variations of the positioning blocks 430 are employed at different locations within the assembly, with corner blocks featuring three-way interlocking surfaces while edge blocks incorporate two-way connections. These components are machined from non-magnetic materials and incorporate features such as alignment pins, keyed interfaces, or surfaces that establish and maintain the correct spatial relationships. They are designed to accommodate thermal expansion while maintaining critical alignments, with materials selected to match the thermal expansion characteristics of the interfacing components.

FIG. 4F illustrates an exploded view of an additional embodiment of the water-cooled coil assembly of the magnetic control assembly. In some embodiments, the water-cooled coil assembly may comprise a first water block 438 and a second water block 440 such that the first water block 438 and the second water block 440 are positioned on opposing sides of the coil 418. The use of multiple water blocks allows a coil 418 of a respective coil mount to be sandwiched between the first water block 438 and the second water block 440 such that both sides of the respective coil 418 are cooled by a respective water block, rather than only a single side being cooled. The second water block 440 may serve as an additional primary thermal interface that directly contacts the coil windings of the respective coil 418 to maximize heat extraction of the magnetic control assembly.

The first water block 438 and the second water block 440 may each include the outer water block portion 420 and the inner water block portion 422. For example, each of the first water block 438 and the second water block 440 may comprise the inlet 412, the outlet 414, the fastening holes 424, the positioning sleeve 426, the coolant channels 428, and/or the positioning blocks 430. In some embodiments, the inner water block portion 422 of the second water block 440 may be a solid plate that does not include the positioning sleeve 426 and/or the positioning blocks 430. In this embodiment, the coolant channels 428, the coolant inlets 412, and the coolant outlets 414 for each of the first water block 438 and the second water block 440 may independently cycle coolant throughout the water-cooled coil assembly to extract heat from both sides of the respective coil 418. The fastening holes 424 may secure the first water block 438 to the second water block 440, ensuring proper alignment and sealing of the coolant channels 428 of both the first water block 438 and the second water block 440.

FIG. 4G illustrates an exploded view of an additional embodiment of the water-cooled coil assembly of the magnetic control assembly. In some embodiments, the water-cooled coil assembly may comprise an encapsulating water block 442 configured to partially or substantially encapsulate the coil 418 of a respective coil mount with coolant by locating the coil 418 within an internal cavity 444. The encapsulating water block 442 may include similar features and functionality of the water-cooled coil assemblies described above, such as the outer water block portion 420. The water-cooled coil assembly may further include a solid plate 443. For example, the encapsulating water block 442 may comprise an internal cavity 444 defined by at least the surfaces of the positioning sleeve 426, the outer water block portion 420, and the solid plate 443. The internal cavity 444 may be sized to receive the entire coil 418. The outer water block portion 420 of the encapsulating water block 442 may further comprise a coolant inlet 412 and a coolant outlet 414, such that the coolant flows within the internal cavity 444 and directly contacts the coil 418 to maximize heat transfer. The outer water block portion 420 and the solid plate 443 may include the fastening holes 424 to secure the outer water block portion 420 and the solid plate 443 together, ensuring proper alignment and sealing of the coil 418 within the internal cavity 444. The inner portion of the positioning sleeve 426 may interface with the positioning components 410 to secure a respective water-cooled coil assembly to a respective coil mount.

The encapsulating water block 442 may further include an opening 445 for feeding additional components through the outer water block portion 420, such as coil windings of the coil 418, pipes, channels, tubing, or other electronic components, or fluidic connection mediums. In some embodiments, the encapsulating water block 442 may incorporate one or more copper tubes or pipes disposed in grooves along the interior surface of the outer water block portion 420, upon which or adjacent to which the coil 418 windings are positioned. For example, such tubing may be fabricated from alternative high-conductivity materials such as aluminum, brass, stainless steel, or thermally conductive composite materials. The copper tubing (or alternative material tubing) may provide an additional high-conductivity path for evacuating heat from the coil 418. Protective coatings such as varnish, epoxy, or polymer layers may be applied to the coil 418 surfaces to reduce the risk of corrosion from direct coolant contact. For example, the protective coating may include multiple layers or may use specialized dielectric coatings or ceramic coatings.

FIG. 4H illustrates a cutaway view of the water-cooled coil assembly disclosed in FIG. 4G. The water-cooled coil assembly may include the coil 418, the encapsulating water block 442, and the solid plate 443. The encapsulating water block 442 may include the outer water block portion 420, the fastening holes 424, the positioning sleeve 426, and the internal cavity 444. As described above, the internal cavity 444 may be defined by at least the surfaces of the positioning sleeve 426, the outer water block portion 420, and the solid plate 443.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as "40 wt.%" is intended to mean "about 40 wt.%".

Several implementations have been described. Nevertheless, it will be understood that various modifications may be made without departing from the spirit and scope of the disclosure. Accordingly, other implementations are within the scope of the following claims.

Clause 1. A portable magnetic control system for microrobots, comprising: a compact power supply unit; an integrated signal generator configured to generate control signals; an amplifier electrically coupled to the compact power supply unit and the signal generator, the amplifier configured to amplify the control signals; a coil assembly electrically coupled to the amplifier, the coil assembly configured to generate magnetic fields based on the amplified control signals; and a cooling system thermally coupled to the coil assembly.

Clause 2. The portable magnetic control system of clause 1, wherein the cooling system is configured to dissipate heat generated by the coil assembly.

Clause 3. The portable magnetic control system of clause 1 and clause 2, wherein the compact power supply unit, signal generator, amplifier, coil assembly, and cooling system are arranged in a system housing to enable field deployment

Clause 4. The portable magnetic control system of clause 1, wherein the power supply unit includes multiple power supplies configured to provide bipolar power.

Clause 5. The portable magnetic control system of clause 1, wherein the signal generator includes a microcontroller programmed to generate arbitrary waveforms.

Clause 6. The portable magnetic control system of clause 1, wherein the amplifier includes linear amplifiers configured in a bridge-tied load configuration.

Clause 7. The portable magnetic control system of clause 1, wherein the coil assembly includes multiple coil pairs arranged orthogonally.

Clause 8. The portable magnetic control system of clause 1, wherein the cooling system includes water blocks thermally coupled to coils of the coil assembly, a pump, and a heat exchanger.

Clause 9. The portable magnetic control system of clause 6, wherein the water blocks include coolant channels formed between inner and outer water block portions.

Clause 10. A method of controlling microrobots using a magnetic control system, comprising: generating control signals using a signal generator; amplifying the control signals using an amplifier; generating magnetic fields using a coil assembly based on the amplified control signals; and dissipating heat generated by the coil assembly using a cooling system.

Clause 11. The method of clause 10, wherein amplifying the control signals includes using linear amplifiers in a bridge-tied load configuration.

Clause 12. The method of clause 10, wherein generating magnetic fields includes generating both static and rotating magnetic fields.

Clause 13. The method of clause 10, further comprising receiving user inputs via a control interface and adjusting the control signals based on the user inputs.

Clause 14. The method of clause 13, wherein the control interface includes software running on an external computer, the software configured to communicate with the signal generator via a USB interface.

Clause 15. The method of clause 14, further comprising adjusting capacitance to optimize power factor based on operating frequency.

Clause 16. A portable magnetic control assembly for microplastic extraction using microrobots, comprising: a housing configured for field deployment in aquatic environments; a bipolar power supply unit disposed within the housing; a signal generator disposed within the housing; an amplifier disposed within the housing and electrically coupled to the bipolar power supply unit and the signal generator; a coil assembly disposed within the housing and electrically coupled to the amplifier, the coil assembly configured to generate magnetic field gradients for applying directional forces to magnetic microrobots; and a cooling system disposed within the housing and thermally coupled to the coil assembly.

Clause 17. The portable magnetic control assembly of clause 16, wherein the signal generator includes a microcontroller programmed to generate arbitrary waveforms.

Clause 18. The portable magnetic control assembly of clause 16, wherein the coil assembly includes multiple coil pairs arranged orthogonally to generate magnetic fields along different axes.

Clause 19. The portable magnetic control assembly of clause 16, wherein the cooling system includes water blocks thermally coupled to coils of the coil assembly, a pump, and a heat exchanger.

Clause 20. The portable magnetic control assembly of clause 16, wherein the water blocks include coolant channels formed between inner and outer water block portions.

Clause 21. The portable magnetic control assembly of clause 20, further comprising a capacitor bank configured to automatically adjust capacitance to optimize power factor based on operating frequency.

Clause 22. The portable magnetic control assembly of clause 16, wherein the power supply is configured to operate at multiple power bands between 300W and 600W.

Clause 23. The portable magnetic control assembly of clause 16, wherein the signal generator is configured to produce control signals with frequencies ranging from DC to several kilohertz.

Clause 24. The portable magnetic control assembly of clause 16, wherein the amplifier has a bandwidth of DC to 10 kHz.

Clause 25. The portable magnetic control assembly of clause 16, wherein the cooling system comprises a closed-loop liquid cooling system.

Clause 26. The portable magnetic control assembly of clause 25, wherein the closed-loop cooling system is capable of dissipating up to 1000W of heat while maintaining coil temperatures below 60°C

1. A portable magnetic control system for microrobots, comprising:
a compact power supply unit configured to provide electrical power;
a signal generator configured to generate control signals;
an amplifier electrically coupled to the compact power supply unit and the signal generator, the amplifier configured to amplify the control signals to generate amplified control signals;
a coil assembly electrically coupled to the amplifier, the coil assembly configured to generate magnetic fields based on the amplified control signals; and
a cooling system thermally coupled to the coil assembly, the cooling system configured to dissipate heat generated by the coil assembly;
wherein the compact power supply unit, the signal generator, the amplifier, the coil assembly, and the cooling system are arranged in a system housing to enable field deployment.

2. The portable magnetic control system of claim 1, wherein the compact power supply unit includes multiple power supplies configured to provide bipolar power.

3. The portable magnetic control system of claim 1, wherein the signal generator includes a microcontroller to generate waveforms.

4. The portable magnetic control system of claim 1, wherein the amplifier includes one or more linear amplifiers configured in a bridge-tied load configuration.

5. The portable magnetic control system of claim 1, wherein the coil assembly comprises a plurality of coil pairs.

6. The portable magnetic control system of claim 1, wherein the cooling system includes one or more water blocks thermally coupled to one or more coils of the coil assembly, one or more pumps, and a heat exchanger.

7. The portable magnetic control system of claim 6, wherein each of the one or more water blocks comprises coolant channels formed between an inner portion and an outer portion.

8. A method of controlling microrobots using a magnetic control system, comprising:
generating control signals using a signal generator;
amplifying the control signals using an amplifier to generate amplified control signals;
generating magnetic fields using a coil assembly based on the amplified control signals; and
dissipating heat generated by the coil assembly using a cooling system.

9. The method of claim 8, wherein generating control signals includes generating waveforms using a microcontroller.

10. The method of claim 8, wherein amplifying the control signals includes using linear amplifiers in a bridge-tied load configuration.

11. The method of claim 8, wherein generating magnetic fields includes generating at least one of static magnetic fields or rotating magnetic fields.

12. The method of claim 8, further comprising receiving user inputs via a control interface and adjusting the control signals based on the user inputs.

13. The method of claim 12, wherein the control interface includes software running on an external computer, the software configured to communicate with the signal generator.

14. The method of claim 13, further comprising adjusting capacitance to optimize power factor based on operating frequency.

15. A portable magnetic control assembly, comprising:
a housing configured for field deployment in aquatic environments;
a bipolar power supply unit disposed within the housing;
a signal generator disposed within the housing;
an amplifier disposed within the housing and electrically coupled to the bipolar power supply unit and the signal generator;
a coil assembly disposed within the housing and electrically coupled to the amplifier, the coil assembly configured to generate magnetic field gradients for applying directional forces to magnetic microrobots; and
a cooling system disposed within the housing and thermally coupled to the coil assembly, the cooling system comprising a closed-loop liquid cooling system.

16. The portable magnetic control assembly of claim 15, wherein the signal generator includes a microcontroller to generate waveforms.

17. The portable magnetic control assembly of claim 15, wherein the coil assembly includes multiple coil pairs arranged orthogonally to generate magnetic fields along different axes.

18. The portable magnetic control assembly of claim 15, wherein the cooling system includes water blocks thermally coupled to one or more coils of the coil assembly, a pump, and a heat exchanger.

19. The portable magnetic control assembly of claim 18, wherein the water blocks include coolant channels formed between inner and outer water block portions.

20. The portable magnetic control assembly of claim 19, further comprising a capacitor bank configured to automatically adjust capacitance to optimize power factor based on operating frequency.
